(19) United States
(12) Patent Application Publication
(10) Pub. No.: US 2023/0225812 A1
(43) Pub. Date: Jul. 20, 2023

(54) SURGICAL ROBOT CALIBRATION DEVICE

(71) Applicant: CMR Surgical Limited, Cambridge (GB)

(72) Inventor: Uresha Kiran Patel, Huntingdon (GB)

(73) Assignee: CMR Surgical Limited, Cambridge, EN (GB)

(21) Appl. No.: 18/247,482

(22) PCT Filed: Sep. 30, 2021
(86) PCT No.: PCT/GB2021/052532

§ 371 Date: Mar. 31, 2023

(30) Foreign Application Priority Data
Oct. 2, 2020 (GB) 2015667.5

(51) Int. Cl. A61B 34/30; A61B 17/00; A61B 90/14

(52) U.S. Cl. CPC A61B 34/30; A61B 17/00; A61B 90/14; A61B 2017/00725; A61B 2034/305; A61B 2034/301

(57) ABSTRACT

A surgical robot calibration device configured to be used when calibrating a surgical robotic system to perform a minimally invasive procedure through a natural orifice, the surgical robotic system comprising a surgical robotic arm and a surgical instrument having a rigid linear shaft, the surgical robot calibration device comprising a resistive spacer configurable to hold a calibration port in a fixed position spaced from the natural orifice, such that when the calibration port is held in the resistive spacer, the surgical instrument is insertable into the natural orifice via the calibration port to enable a fulcrum about which the surgical instrument pivots whilst the surgical instrument is inserted into the calibration port to be determined.

### BACKGROUND

This invention relates to a surgical robot calibration device.

FIG. 1 shows a surgical robotic system 100 performing a minimally invasive procedure on a patient 102. The patient 102 is positioned on an operating table 103. In FIG. 1, the procedure is being performed through a natural orifice of the patient 102. The natural orifice is the patent's mouth. A minimally invasive procedure performed through the mouth is typically termed a transoral surgery, and is often performed, for example, to remove tumours from the throat.

The surgical robot 100 comprises a robotic arm 101. The robotic arm 101 comprises a plurality of joints 104 by which the configuration of that robotic arm can be altered. The robotic arm 101 comprises an attachment for a surgical instrument 106 at its distal end. The surgical instrument has an end effector at its distal end for performing aspects of the minimally invasive procedure. The surgical instrument could, for example, be a cutting or grasping device, or an imaging device (such as an endoscope).

The configuration of the robotic arm 101 may be remotely controlled in response to inputs received at a remote surgeon console 120. A surgeon may provide inputs to the remote console 120. The remote surgeon console comprises one or more surgeon input devices. For example, these may take the form of a hand controller and/or foot pedal.

A control system 124 connects the surgeon console 120 to the surgical robotic arm 101. The control system receives inputs from the surgeon input device(s) and converts these to control signals to move the joints 104 of the robotic arm and the surgical instrument 106. The control system 124 sends these control signals to the robot, where the corresponding joints are driven accordingly. In order for the control system to control the surgical robotic arm correctly and safely, the surgical robot must be calibrated.

### SUMMARY OF THE INVENTION

According to a first aspect of the invention there is provided a surgical robot calibration device for calibrating a surgical robotic system to perform a minimally invasive procedure through a natural orifice, the surgical robotic system comprising a surgical robotic arm and a surgical instrument having a rigid linear shaft, the surgical robot calibration device comprising a resistive spacer configurable to hold a calibration port in a fixed position spaced from the natural orifice, such that when the calibration port is held in the resistive spacer, the surgical instrument is insertable into the natural orifice via the calibration port.

The resistive spacer may be configured to be received at one end in the natural orifice, and comprise an aperture at an opposing end for receiving the calibration port.

The resistive spacer may be configured to be received at one end in the natural orifice and comprise a surface spaced from that end having one or more apertures each configured to receive a calibration port.

The resistive spacer may be configured to be received at one end in the natural orifice and comprise a surface spaced from that end that is pierceable so as to receive a calibration port.

The resistive spacer may be curved so as to define a substantially domed three-dimensional shape.

The resistive spacer may be configured to bridge the natural orifice.

The resistive spacer may comprise a calibration port attachment that is movably mounted with respect to the resistive spacer.

The moveably mounted calibration port attachment may be mounted on one or more moveable rails such that, when the resistive spacer is bridging the natural orifice, the calibration port attachment is moveable in a plane above the natural orifice.

The calibration port attachment may be securable in position relative to the resistive spacer such that, in use, the calibration port is held in the fixed position.

The resistive spacer may comprise a surface having one or more apertures each configured to receive a calibration port.

The resistive spacer may comprise a surface that is pierceable so as to receive a calibration port.

The natural orifice may be a patient's mouth and the resistive spacer may be configured to bridge the patient's head so as to bridge the mouth.

The resistive spacer may extend from a base at its proximal end, comprise a plurality of joints by which the configuration of that resistive spacer can be altered and comprise an attachment for the calibration port at its distal end.

According to a second aspect of the invention there is provided a surgical robot calibration device for calibrating a surgical robotic system to perform a minimally invasive procedure through a natural orifice, the surgical robotic system comprising a surgical robotic arm and a surgical instrument having a rigid linear shaft, the surgical robot calibration device comprising a resistive spacer having formed integrally therewith a calibration port, the resistive spacer being configured to retain the calibration port at a fixed position spaced from the natural orifice, such that the surgical instrument is insertable into the natural orifice via the calibration port.

The resistive spacer may be configured to be received at one end in the natural orifice, and the calibration port may be formed integral therewith at an opposing end.

The resistive spacer may be configured to be received at one end in the natural orifice and comprise a surface spaced from that end having one or more calibration ports formed integral therewith.

The resistive spacer may be curved so as to define a substantially domed three-dimensional shape.

The resistive spacer may be configured to bridge the natural orifice.

The calibration port may be movable with respect to the resistive spacer.

The calibration port may be formed integrally with a moveable rail such that, when the resistive spacer is bridging the natural orifice, the calibration port is moveable in a plane above the natural orifice.

The calibration port may be securable in position relative to the resistive spacer such that, in use, the calibration port is held in the fixed position.

The resistive spacer may comprise a surface having one or more calibration ports formed integrally therewith.

The natural orifice may be a patient's mouth and the resistive spacer may be configured to bridge the patient's head so as to bridge the mouth.

The resistive spacer may extend from a base at its proximal end, comprise a plurality of joints by which the configuration of that resistive spacer can be altered and have the calibration port formed integrally therewith at its distal end.

The natural orifice may be a mouth and the resistive spacer may comprise an attachment for a mouth retractor.

The mouth retractor may be detachable from the attachment.

The resistive spacer may be elastically deformable, and the attachment may comprise a lip, such that: with the resistive spacer in a first configuration, the lip attaches the mouth retractor to the resistive spacer; and with the resistive spacer in a second, deformed, configuration, the mouth retractor can pass over the lip in order to detach the mouth retractor from the resistive spacer.

The resistive spacer may be capable of resisting external forces applied to the calibration port by the surgical instrument such that, in use, the calibration port is maintained in the fixed position.

The resistive spacer may be capable of resisting external forces of at least 2 Newtons applied to the calibration port by the surgical instrument.

According to a third aspect of the invention there is provided a method of calibrating a surgical robotic system to perform an invasive procedure via a natural orifice using the surgical robot calibration device as claimed in any preceding claim, the surgical robotic system comprising a surgical robotic arm having a series of joints by which the configuration of the robotic arm can be altered, the series of joints extending from a base at a proximal end of the surgical robotic arm to a surgical instrument having a rigid linear shaft attached at a distal end of the surgical robotic arm, and the method comprising determining a fulcrum about which the surgical instrument pivots when the configuration of the robotic arm is altered whilst the surgical instrument is inserted into the calibration port.

### BRIEF DESCRIPTION OF THE DRAWINGS

The present invention will now be described by way of example with reference to the accompanying drawings. In the drawings:

FIG. 1 shows a surgical robotic system.

FIG. 2 shows an example of a surgical robotic arm.

FIG. 3 is a flow diagram showing the steps of a surgical robot calibration process.

FIG. 4a shows a first example surgical robot calibration device.

FIG. 4b shows a second example surgical robot calibration device.

FIG. 4c shows a third example surgical robot calibration device.

FIG. 4d shows the first example surgical robot calibration device.

FIG. 4e shows a fourth example surgical robot calibration device.

FIG. 4f shows a fifth example surgical robot calibration device.

FIGS. 5a, 5b and 5c show an example resistive spacer comprising an attachment for a detachable mouth retractor.

FIG. 6a shows a sixth example surgical robot calibration device.

FIG. 6b shows a seventh example surgical robot calibration device.

FIG. 7 shows a eighth example surgical robot calibration device.

### DETAILED DESCRIPTION OF THE DRAWINGS

The following description is presented to enable any person skilled in the art to make and use the invention, and is provided in the context of a particular application. Various modifications to the disclosed embodiments will be readily apparent to those skilled in the art.

The general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

Surgical Robotic Arm

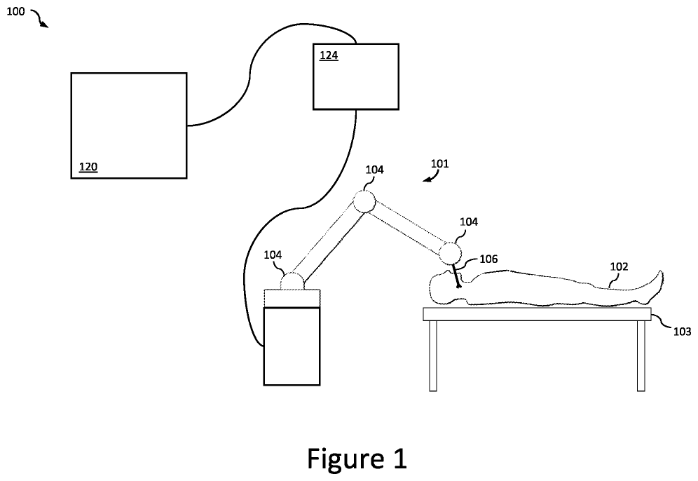

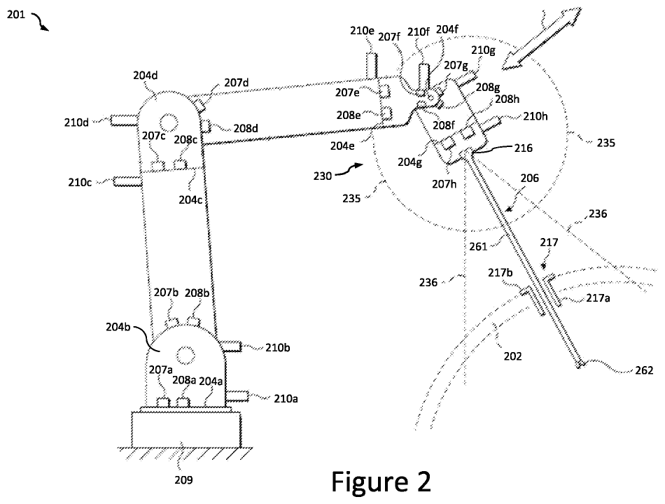

FIG. 2 shows an example of a robotic arm 201. The robotic arm 201 may be comprised within a surgical robotic system, such as the surgical robotic system shown in FIG. 1. Although the surgical robotic system shown in FIG. 1 comprises one surgical robotic arm, it is to be understood that a surgical robotic system may comprise a plurality of surgical robotic arms.

The robotic arm 201 comprises a base 209. The robotic arm has a series of rigid arm members. Each arm member in the series is joined to the preceding arm member by a respective joint 204a-g. Joints 204a-e and 204g are revolute joints. Joint 204f is composed of two revolute joints whose axes are orthogonal to each other, as in a Hooke's or universal joint. Joint 204f may be termed a “wrist joint”. A robotic arm could be jointed differently from the robotic arm of FIG. 2. For example, joint 204d could be omitted and/or joint 204f could permit rotation about a single axis. The robotic arm could include one or more joints that permit motion other than rotation between respective sides of the joint, such as a prismatic joint by which an instrument attachment can slide linearly with respect to more proximal parts of the robotic arm.

The joints are configured such that the configuration of the robotic arm can be altered allowing the distal end 230 of the robotic arm to be moved to an arbitrary point in a three-dimensional working volume illustrated generally at 235. One way to achieve that is for the joints to have the arrangement illustrated in FIG. 2. Other combinations and configurations of joints could achieve a similar range of motion, at least within the zone 235. There could be more or fewer arm members.

The robotic arm 201 comprises a series of motors 210a-h. With the exception of the compound joint 204f, which is served by two motors, each motor is arranged to drive rotation about a respective joint of the robotic arm. The motors are controlled by a control system (such as control system 124 shown in FIG. 1). The control unit comprises a processor and a memory. The memory stores, in a non-transient way, software code that can be executed by the processor to cause the processor to control the motors 210a-h in the manner described herein.

The robotic arm 201 may comprise a series of sensors 207a-h and 208a-h. These sensors may comprise, for each joint, a position sensor 207a-h for sensing the rotational position of the joint and a force sensor 208a-h for sensing forces (such as torque) applied about the joint's rotation axis. Compound joint 204f may have two pairs of sensors. One or both of the position and force sensors for a joint may be integrated with the motor for that joint. The outputs of the sensors are passed to the control system where they form inputs for the processor.

The distal end of the robotic arm 230 has an attachment 216 by means of which a surgical instrument 206 can be releasably attached. The surgical instrument has a rigid linear shaft 261. The surgical instrument has an end effector 262 at the distal end of the shaft. The end effector 262 consists of a device for engaging in a procedure, for example a cutting, grasping or imaging device. As described herein, terminal joint 204g may be a revolute joint. The surgical instrument 206 and/or the attachment 216 may be configured so that the instrument extends linearly parallel with the rotation axis of the terminal joint 204g of the robotic arm. In this example the instrument extends along an axis coincident with the rotation axis of joint 204g.

Joints 204e and 204f of the robotic arm are configured so that with the distal end of the robotic arm 230 held at an arbitrary location in the working volume 235 the surgical instrument 206 can be directed in an arbitrary direction within a cone. Such a cone is illustrated generally at 236. One way to achieve that is for the terminal part of the arm to comprise the pair of joints 204e and 204f whose axes are mutually arranged as described above. Other mechanisms can achieve a similar result. For example, joint 204g could influence the attitude of the instrument if the instrument extends in a direction which is not parallel to the axis of joint 204g.

For some types of minimally invasive procedure, the surgical instrument 206 may be inserted into the patient's body through a synthetic port 217. For example, the minimally invasive procedure may be performed within the patient's abdomen. The port 217 may comprise a passageway 217a. The passageway 217a may pass through the outer tissues 202 of the patient so as to limit disruption to those tissues as the surgical instrument is inserted and removed, and as the instrument is manipulated within the patient's body. The port 217 may comprise a collar 217b. The collar 217b may prevent the port 217 being inserted too far through the outer tissues 202 of the patient.

For other types of minimally invasive procedure, the surgical instrument may be inserted directly into the patient's body through a natural orifice. For example, the minimally invasive procedure may be performed in the patient's throat, and the natural orifice may be the patient's mouth (e.g. as shown in FIG. 1).

Calibration Process

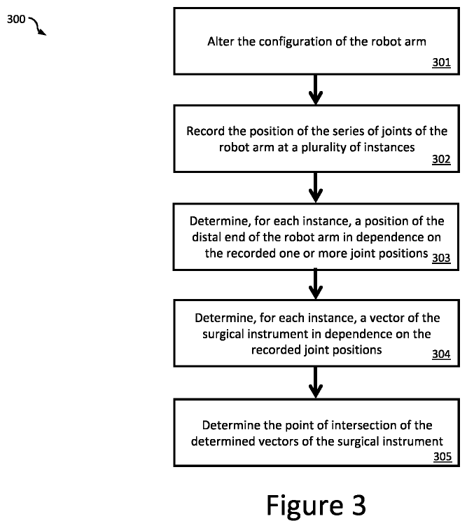

The surgical robot is calibrated prior to performing a minimally invasive procedure. During calibration, the control system (e.g. such as control system 124 shown in FIG. 1) of the robotic arm 201 determines the fulcrum about which the surgical instrument pivots when the configuration of the robotic arm is altered whilst the surgical instrument is inserted into the port or the natural orifice. The control system determines said fulcrum by means of a calibration process that is performed whilst the surgical instrument 206 is inside the port or natural orifice. FIG. 3 is a flow diagram showing the steps of such a calibration process.

Whilst the surgical instrument is inside the port or natural orifice, the configuration of the robotic arm is altered 301. The configuration of the robotic arm 301 is altered by the application of external forces directly onto the robotic arm. For example, a member of the bedside team (e.g. an operating room nurse) may apply forces directly to the robotic arm (e.g. by pushing a joint of the robotic arm). During calibration, the control system (such as control system 124 shown in FIG. 1) controls the robotic arm to maintain a position in which it is placed by means of external forces applied directly to the robotic arm.

To achieve this, the control system receives inputs from the position and force sensors 207a-h and 208a-h. From the position sensors the control system can determine the current configuration of the robotic arm. The control system stores for each element of the robotic arm, and the surgical instrument, its mass, the distance of its centre of mass from the preceding joint of the robotic arm and the relationship between the centre of mass and the positional output of the position sensor for the preceding joint. The current configuration of the robotic arm could be inferred by other means. For example, camera-based positioning systems may be used to track points in space, such as fiducial markers attached to the robotic arm. This technique could be used to determine the joint angles. Other techniques include inferring the position of a joint using a current sensors. For example, the position of a joint can be inferred from the amount of current passing through the motor and assuming a given relationship to be constant.

Using that information, the control system models the effect of gravity on the components of the robotic arm for the current configuration of the robotic arm and estimates a force (e.g. a torque) due to gravity on each joint of the robotic arm. The processor then drives the motor 210a-h of each joint to apply a force (e.g. a torque) that will exactly oppose the calculated gravitational force. With this control strategy an operator (e.g. an operating room nurse) can push or pull any part of the robotic arm to a desired position, and the part will stay in that position notwithstanding the effect of gravity on it and on any parts depending from it. A force on the robotic arm may result in a torque about multiple joints. The control system can be programmed to decide to prioritise certain ones of the joints for neutralising the torque. In examples, some joints could be locked in position and others could move compliantly, the position of a given link or point in space could be prioritized rather than sets of joints.

Each motor 210a-h may be controlled in response to the force (e.g. torque) measured about the respective joint. When the measured force at a joint is adjusted for gravity, any remaining sensed force represents a force applied by an external force (e.g. due to a push or pull on the robotic arm). In response to that force the control system may control the respective motor 210a-h so as to alter the configuration of the robotic arm. For example, this may be achieved by controlling the motors 210a-h to move their respective joints 204a-g in a direction so as to reduce the measured force, and at a rate dependant on the magnitude of the measured force. In this way, the member of the bedside staff may feel that that the robotic arm is moving freely in response to the force they are applying—when in fact it is the motors of the robotic arm driving the movement.

During calibration, the robotic arm can be moved generally transversely to the rigid linear shaft 261 of the instrument 206. The configuration of the robotic arm may be altered such that the distal end of the robotic arm is moved in two dimensions: e.g. with (i) components parallel to a direction that is transverse to the instrument shaft 261 and also with (ii) components orthogonal to that direction but transverse to the instrument shaft 261. To do this, the operator (e.g. a member of the bedside team) may gyrate the distal end of the robotic arm about a point generally aligned with the natural axis of the passageway of the port or natural orifice. This causes the port or natural orifice to apply a lateral force on the instrument shaft 216. That force is accommodated by motion about the joint 204f.

As the configuration of the robotic arm is being altered, the position sensors 207a-h record the position of each joint of the robotic arm. The position sensors 207a-h record 302 the positions of each joint of the robotic arm at a plurality of instances. That is, the position sensors 207a-h record the positions of each joint of the robotic arm at a plurality of points in time. Position information may be recorded irregularly or at predetermined intervals, e.g. every 0.5 seconds. The position sensors provide the recorded position information to the control system.

The control system uses this received information to determine: (a) the position of the distal end of the robotic arm relative to the base and (b) the vector of the instrument shaft 261 relative to the distal end of the robotic arm. Position (a) and vector (b) may be termed a data pair. The control system may determine a data pair for each instance at which the position sensors recorded position information. That is, for each instance, the control system determines 303 a position of the distal end of the robotic arm in dependence on the recorded one or more joint positions. In addition, for each instance, the control system determines 304 a vector of the surgical instrument from the determined position of the distal end of the second robotic arm in dependence on the recorded joint positions. Since the rigid linear axis of the instrument shaft 261 passes through the port or natural orifice, the port or natural orifice lies along that vector. As the distal end of the robotic arm is moved, the control system calculates multiple pairs of distal end positions and instrument shaft vectors. Those vectors all converge, from their respective distal end position, on the natural rotation centre of the port or natural orifice. By collecting a series of those data pairs and then solving for the mean location where the instrument shaft vectors converge the control system estimates the location of the port or natural orifice relative to the robotic arm. That is, the control system determines 305 the point of intersection of the determined vectors of the surgical instrument so as to determine the fulcrum. The control system then stores the determined fulcrum in non-transient form in memory for later use.

The number of data pairs required to determine the fulcrum with acceptable precision depends on factors such as the accuracy of the robotic arm's position sensors and the extent to which the operator moves the arm laterally during the calibration process. The control system may determine that the fulcrum has been estimated adequately once sufficient coherent measurements have been gathered such that the variance between estimates of the fulcrum derived using successive measurements has reduced below a predefined level.

After determining the fulcrum, the robotic arm can be used to perform a minimally invasive procedure. During a minimally invasive procedure, the configuration of the robotic arm may be remotely controlled in response to inputs received at a remote surgeon console (such as remote surgeon console 120 shown in FIG. 1). During the minimally invasive procedure, the operator (e.g. a surgeon) uses the remote surgeon console to signal a desired position of the end effector 262. The control system (such as control system 124 shown in FIG. 1) determines a configuration of the joints of the robotic arm that will result in the end effector 262 being placed in that position. The control system uses the determined fulcrum to assist in controlling the configuration of the robotic arm when the robotic arm is operating in the surgical mode. The control system is configured, e.g. by means of the software stored in memory, to select a configuration of the arm for which both (i) the end effector 262 is at the desired position and (ii) the rigid shaft 261 of the instrument 206 passes through the determined fulcrum, and to move the arm to that configuration. In that way the end effector 262 can be provided at the desired position with relatively little disruption to the outer tissues of the patient.

Surgical Robot Calibration Device

For minimally invasive procedures performed within a patient's abdomen, each surgical instrument used for that procedure is inserted into the patient's abdomen through a synthetic port (e.g. port 217 in FIG. 2) placed in an incision in the patient's abdomen. The purpose of the port in such procedures is to hold the incision open, so as to ensure that surgical instruments can be inserted and removed from the abdomen with limited disruption to the incision. Additionally, during the calibration process as described herein, the port protects the outer tissues of the patient's abdomen from damage by the instrument shaft. This is because the instrument shaft interacts with and exerts forces onto the passageway of the port whist it is being gyrated during the calibration process, rather than interacting directly with the incision in the patient's abdomen. Further, when performing minimally invasive procedures within a patient's abdomen, the abdomen is typically inflated such that the amount of empty space within the abdomen is increased. With the abdomen inflated, ample space is provided for the end effector of the surgical instrument to move during the calibration process without interacting with, or causing damage to, tissue within the abdomen.

Minimally invasive procedures can also be performed through a natural orifice, such as the mouth. Synthetic ports (such as port 217 in FIG. 2) are not typically used when performing minimally invasive procedures via natural orifices. This is because, as a natural orifice is being used as an access point, there is no need for a port to hold a tissue incision open. Whilst a port is not required during the minimally invasive procedure itself, there can be a number of problems associated with performing the calibration process described herein with the surgical instrument inserted through the natural orifice. First, without a port present, the instrument shaft interacts with and exerts forces directly onto the natural orifice whist it is being gyrated during the calibration process. This can cause damage about the opening of the natural orifice, such as chipping of the teeth. Second, there is a limited amount of space available within natural orifices such as the mouth, and so the movement of the instrument during the calibration process can cause the end effector of the surgical instrument to impinge on the interior of the natural orifice. This can cause damage to the interior of the natural orifice, such as bruising and/or lacerations to the interior of the mouth. Third, as the internal surfaces of natural orifices such as the mouth are non-uniform (e.g. by contrast to a cylindrical synthetic port), it can be more difficult for the processor to accurately determine the fulcrum about which the surgical instrument pivots when the configuration of the surgical robotic arm is altered. As a result, the processor may need to acquire more data pairs in order to estimate the fulcrum location with acceptable precision. This means that the calibration process may take longer to complete—which can further increase the amount of damage caused to the natural orifice during the calibration process.

Described herein is a surgical robot calibration device for calibrating a surgical robot to perform a minimally invasive procedure through a natural orifice. To address one or more of the problems identified in the preceding paragraph, the surgical robot calibration device enables the calibration process to be performed using a calibration port held in a fixed position spaced from the natural orifice. In this way, the instrument shaft interacts with and exerts forces directly onto the calibration port whist it is being gyrated during the calibration process—thus avoiding damage about the opening of the natural orifice, and making it easier to accurately determine the fulcrum position.

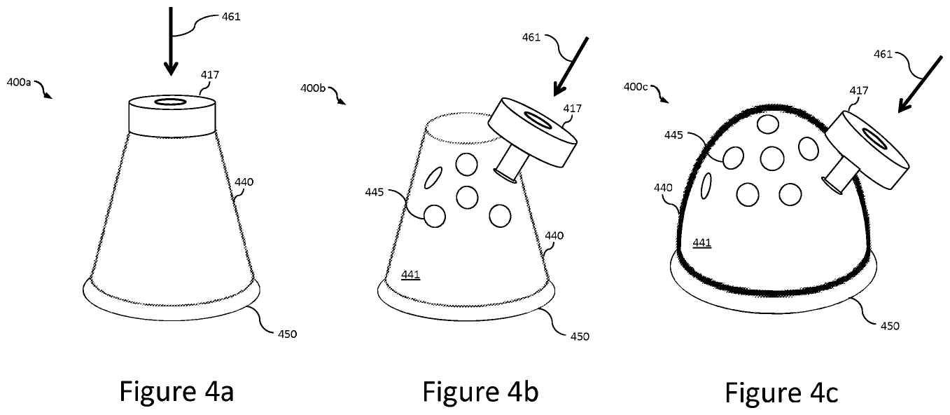

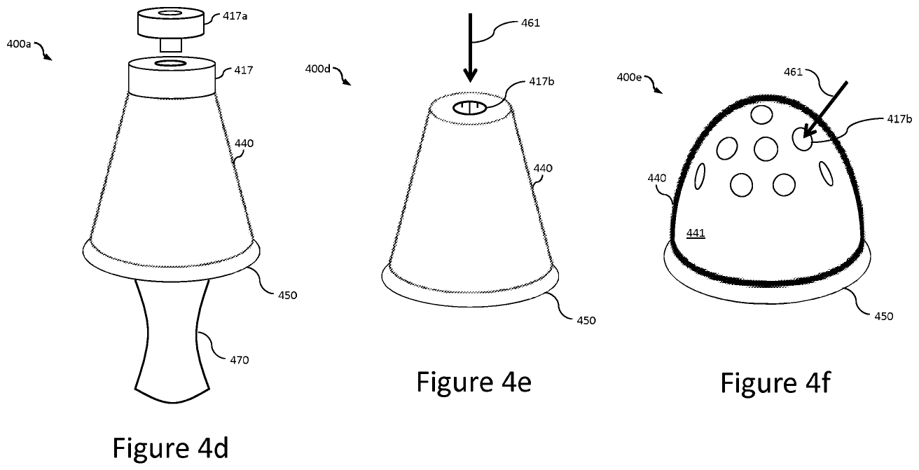

FIG. 4a shows a first example surgical robot calibration device. The surgical robot calibration device 400a comprises a resistive spacer 440 that is configured to hold a calibration port 417. During the calibration process, one end of the resistive spacer 440 is received in the natural orifice. In examples where the natural orifice is a mouth, that end of the resistive spacer may act as a mouth retractor 450 for holding the mouth open, or comprise an attachment for a mouth retractor 450. The calibration port 417 is received in an aperture at an opposing end of the resistive spacer 440. In this way, the resistive spacer 440 holds the calibration port 417 in a fixed position spaced from the natural orifice. During the calibration process, the surgical instrument is insertable into the natural orifice via the calibration port 417, e.g. in the direction shown by arrow 461.

After the calibration process as described herein has been completed, the resistive spacer 440 and the calibration port 417 can be removed such that the minimally invasive procedure can be performed through the natural orifice. Performing the calibration process in this manner may result in the determined fulcrum location being spaced from the natural orifice, however this spacing can be accounted for by the control system when controlling the surgical robotic arm.

The calibration port 417 may be a standard surgical port, for example, of the type used in minimally invasive procedures performed within a patient's abdomen. The calibration port 417 may have the same features as synthetic port 217 shown in FIG. 2. That is, the calibration port may comprise a collar. The calibration port 417 rests on the collar when placed in the aperture in the resistive spacer 440. The calibration port 417 may comprise a passageway. The passageway enables the passage of surgical instruments through the calibration port 417. The passageway may be 5 mm or 10 mm in diameter. These are standard port passageway dimensions. Surgical instruments such as cutters and graspers may be calibrated using a calibration port having a passageway of 5 mm in diameter. Surgical instruments such as endoscopes may be calibrated using a calibration port having a passageway of 10 mm in diameter. The calibration port 417 may comprise an attachment for a reduction cap (e.g. 417a in FIG. 4d) which acts to reduce the diameter of the passageway. For example, the passageway may have a diameter of 10 mm, and the reduction cap may have a diameter of 5 mm. In this way, the same calibration port can be used to calibrate surgical instruments such as endoscopes without the reduction cap attached, and surgical instruments such as cutters and graspers with the reduction cap attached. The passageway and/or reduction cap may be of any other suitable dimension.

In the first example shown in FIG. 4a, the resistive spacer 440 has a frustoconical shape. That is, the resistive spacer 440 has the shape of a frustum or a cone. It is to be understood that the resistive spacer could be any other suitable shape, such as an oval based frustum, a square or rectangular based frustum, a cuboid, or a dome.

The dimensions of the opposing ends of the resistive spacer may be dictated by the dimensions of the calibration port and the natural orifice. For example, where the natural orifice is a mouth, the diameter of the end configured to be received in the mouth may be between 25 mm to 90 mm. A range of differently dimensioned resistive spacers may be provided such that an appropriately dimensioned resistive spacer can be selected in dependence on the size of the patient's mouth—or other natural orifice through which a minimally invasive procedure is to be performed. The diameter of the opposing end may be in the range of 13 mm to 15 mm, and comprise an aperture suitably dimensioned to receive the calibration port. The vertical height of the resistive spacer 440 may be in the range of 30 mm to 100 mm. The resistive spacer should be tall enough so as to enable a surgical instrument to be inserted past the fulcrum about which it will pivot during the calibration process, but not so tall that the resistive spacer is unstable during the calibration process.

The resistive spacer 440 may be hollow. In this way, the interior of the resistive spacer 440 provides space for the end effector of the surgical instrument to move during the calibration process. That is—whilst the geometry of the resistive spacer 440 is such that the end effector of the surgical instrument could be inserted into the natural orifice via the calibration port when the calibration port is held in the resistive spacer 440—during the calibration process the end effector of the surgical instrument need not actually be inserted into the natural orifice. The calibration process could be performed with the surgical instrument inserted into the surgical robot calibration device only so far that the end effector is within the interior of the resistive spacer 440. In this way, the movement of the instrument during the calibration process does not cause the end effector of the surgical instrument to impinge on the interior of the natural orifice—and so damage to the natural orifice can be avoided.

The resistive spacer 440 is capable of resisting external forces applied to the calibration port by the surgical instrument such that, during the calibration process, the calibration port is maintained in the fixed position. That is, as described herein, the rigid linear shaft of the surgical instrument interacts with and exerts forces onto the passageway of the port whist it is being gyrated during the calibration process. Said forces can be in the region of 2 to 3 Newtons. The resistive spacer is capable of resisting external forces of at least 2 Newtons applied to the calibration port by the surgical instrument. In order to achieve this, the resistive spacer may be constructed from a material capable of resisting said forces, such as stainless steel, medical grade silicone, or other medical grade plastics (e.g. polycarbonates, polystyrene, acrylic or polyurethane). Stainless steel is additionally advantageous as it is autoclavable (so as to be sterilised), biocompatible, and corrosion resistant. Silicone is additionally advantageous as it is sterilisable, biocompatible, and cost-effective. Polycarbonates are also often autoclavable and biocompatible, and may be further advantageous as they can be transparent or translucent—so as to enable visibility through the resistive spacer. Polystyrene is also typically sterilisable, e.g. using a process such as ethylene oxide gas sterilisation.

The resistive spacer 440 and/or the calibration port 417 may be sterilisable and re-usable, or may be disposable after a single use.

FIG. 4b shows a second example surgical robot calibration device 400b. The resistive spacer 440 is configured to be received at one end in the natural orifice. Relative to the first example shown in FIG. 4a, in the second example shown in FIG. 4b the resistive spacer 440 comprises a surface 441 spaced from that end having a plurality of apertures 445 each configured to receive a calibration port 417. Each of the plurality of apertures are at different fixed distances from the natural orifice. Each of plurality of apertures are also angled differently relative to the natural orifice. This means that, in use, the resistive spacer can be placed in the natural orifice and, regardless of its precise orientation, an appropriate aperture can be selected for use in calibrating a surgical robot arm. Further, multiple surgical robotic arms each comprising a surgical instrument to be used in the minimally invasive procedure can be calibrated using the same surgical robot calibration device 400b—where each instrument approaches the natural orifice from a different direction and is calibrated to its own unique fulcrum.

FIG. 4c shows a third example surgical robot calibration device. As in the second example shown in FIG. 4b, in the third example the resistive spacer 440 is configured to be received at one end in the natural orifice, and comprises a surface 441 spaced from that end having a plurality of apertures 445 each configured to receive a calibration port 417. In the third example, the surface 441 of the resistive spacer 440 is curved so as to define a domed three-dimensional shape. This shape is advantageous as it has a greater surface area than a frustoconical shape of the same height. This means that the surface 441 may comprise a greater number of apertures 445 for receiving calibration ports—meaning that the surgeon is allowed more flexibility in where the calibration port is placed. In addition, owing to the geometry of a dome shape, those apertures in the curved surface 441 are naturally angled towards the end of the resistive spacer 440 that is configured to be received in the natural orifice.

In another example, the resistive spacer 440 may be configured to be received at one end in the natural orifice and may comprise a surface 441 spaced from the end that is pierceable so as to receive a calibration port. That is, the surface 441 may initially comprise no apertures configured to receive a calibration port, but may be pierceable by a process such as cutting or drilling so as to create an aperture for receiving a calibration port 417. In this way, a surgeon or member of the operating room staff can determine the precise position in which the calibration port 417 is to be held, rather than selecting from one or more predetermined positions as in the examples shown in FIGS. 4a, 4b and 4c. In other words, the resistive spacer 440 is configurable to hold a calibration port 417 in a fixed position spaced from the natural orifice.

In order for the surface 441 of the resistive spacer 440 to be pierceable in this way it may be constructed from a material such as silicone (e.g. pierceable by cutting using a scalpel), stainless steel (e.g. pierceable by drilling using a power tool), or any other suitable material.

As described herein, in examples where the natural orifice is a mouth, the end of the resistive spacer 440 that is configured to be received in the mouth may act as a mouth retractor 450 for holding the mouth open.

In an example, the resistive spacer 440 may be elastically deformable. The portion of the resistive spacer acting as a mouth retractor 450 can be elastically deformed under the action of an external force in order to be placed in the mouth, such that when that external deforming force is removed it will exert an outward force to hold the mouth open. The external deforming force could be applied by hand by a surgeon or a member of the operating room staff. Alternatively, a tool such as a calliper or clamp may be provided to apply the deforming force to the resistive spacer. The force required to elastically deform the resistive spacer may be in excess of 8 Newtons. In order for the resistive spacer 440 to be elastically deformable in this way it may be constructed from a material such as silicone. After the calibration process has been completed the resistive spacer 440 including the portion acting as a mouth retractor 450 may be elastically deformed so as to be removed from the mouth—leaving the mouth empty.

As described herein, in examples where the natural orifice is a mouth, the end of the resistive spacer 440 that is configured to be received in the mouth may comprise an attachment for a mouth retractor 450. The mouth retractor 450 may be detachable from the mouth retractor attachment. For example, the mouth retractor 450 may be screw fit onto the resistive spacer 440, or may be attached to the resistive spacer by one or more clips. In these examples, after the calibration process has been completed the resistive spacer 440 may be removed, leaving the mouth retractor 450 in place in the mouth. In this way, the detachable mouth retractor 450 can be used to hold the mouth open during the minimally invasive procedure.

In an example, the detachable mouth retractor 450 may comprise a ratchet mechanism that can be used to increase or decrease the diameter of the mouth retractor 450—so as to fit different sized mouths.

FIG. 4e shows a fourth example surgical robot calibration device. Relative to the first example shown in FIG. 4a, in the fourth example shown in FIG. 4e the resistive spacer 440 has a calibration port 417b formed integrally therewith. That is, the calibration port 417b may not be removable from the resistive spacer. For example, the calibration port 417b may be a through hole in a surface of the resistive spacer. The resistive spacer 440 shown in FIG. 4e is configured to retain the calibration port at a fixed position spaced from the natural orifice. During the calibration process, the surgical instrument is insertable into the natural orifice via the calibration port 417b, e.g. in the direction shown by arrow 461.

The calibration port 417b may comprise a passageway. The passageway enables the passage of surgical instruments through the calibration port 417b. During the calibration process, the rigid linear shaft of the surgical instrument interacts with and exerts forces onto the passageway of the port whist it is being gyrated during the calibration process. The passageway may be reinforced relative to the remainder of the resistive spacer so as to resist external forces applied to the calibration port by the surgical instrument. For example, the thickness of the resistive spacer may be greater about the calibration port 417b than about the remainder of the resistive spacer. The passageway may be 5 mm or 10 mm in diameter.

The calibration port 417b may comprise an attachment for a reduction cap (e.g. 417a in FIG. 4d) which acts to reduce the diameter of the passageway.

The fourth example surgical robot calibration device described with reference to FIG. 4e may have each of the other features and/or advantages of the first example surgical robot calibration device described with reference to FIG. 4a.

It is to be understood that any of the resistive spacers described herein may have one or more calibration ports formed integrally therewith as described with reference to FIG. 4e instead of, or in addition to, being configurable to hold a calibration port. For example, FIG. 4f shows a fifth example surgical robot calibration device. Relative to the third example shown in FIG. 4c, in the fifth example shown in FIG. 4c the resistive spacer 440 comprises a surface 441 having a plurality of calibration ports 417b formed integrally therewith. Each of the plurality of calibration ports 417b may have the same features as the calibration port 417b described with reference to FIG. 4e. The fifth example surgical robot calibration device may have each of the other features and/or advantages of the third example surgical robot calibration device described with reference to FIG. 4c.

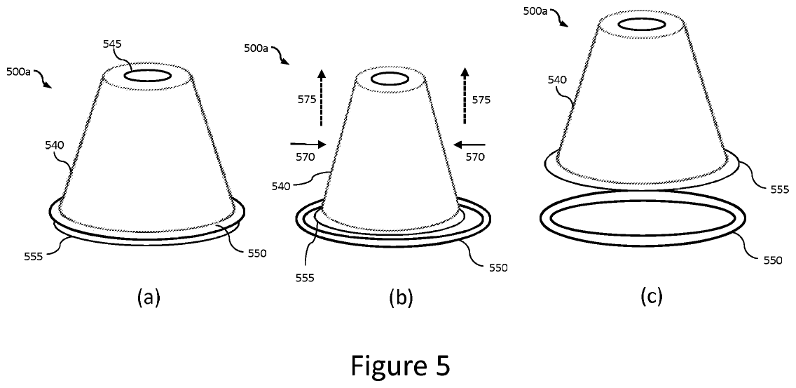

FIG. 5 shows an example resistive spacer comprising an attachment for a detachable mouth retractor. For simplicity, the calibration port is not shown in FIG. 5. It is to be understood that the concepts described with reference to FIG. 5 could be applied to any of the examples shown in FIG. 4a, 4b, 4c, 4e or 4f. The resistive spacer 540 shown in FIG. 5 can be elastically deformed under the action of an external force. The resistive spacer 540 comprises an attachment 555 for a mouth retractor 550. The attachment 555 is a lip, preferably a circumferential lip. With the resistive spacer 540 in a first configuration (as shown in (a)), the lip attaches the mouth retractor 550 to the resistive spacer 540. With the resistive spacer 540 in a second, elastically deformed, configuration (as shown in (b)), the mouth retractor 550 can pass over the lip in order to detach the mouth retractor 550 from the resistive spacer 540. The external deforming force could be applied by hand by a surgeon or a member of the operating room staff—e.g. in directions 570. Alternatively, a tool such as a calliper or clamp may be provided to apply the deforming force to the resistive spacer. The force required to elastically deform the resistive spacer may be in excess of 8 Newtons. In order for the resistive spacer 440 to be elastically deformable in this way it may be constructed from a material such as silicone. After the calibration process has been completed, the resistive spacer 540 may be removed, e.g. in direction 575, leaving the mouth retractor 550 in place in the mouth—as shown in (c).

The portion of the resistive spacer acting as a mouth retractor 450, or the detachable mouth retractor 550, may comprise a tongue depressor (e.g. 470 in FIG. 4d) for holding the patient's tongue against the floor of the mouth during the calibration process and/or the following minimally invasive procedure.

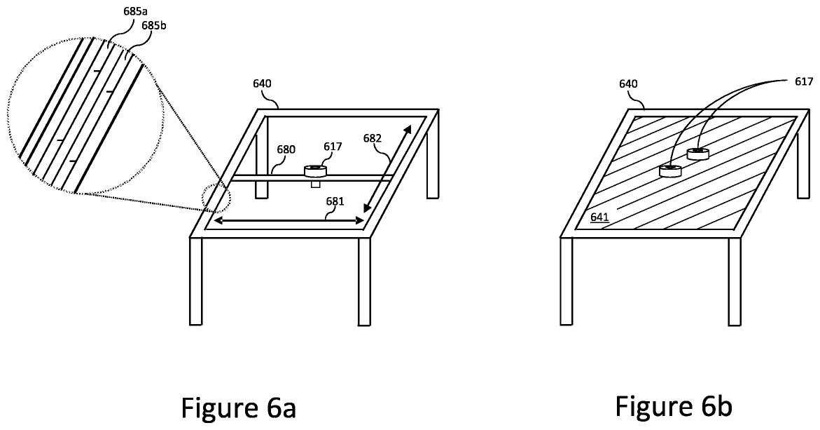

FIG. 6a shows a sixth example surgical robot calibration device. In this example, the resistive spacer 640 is configured to bridge the natural orifice—rather than be received in the natural orifice as described with reference to FIGS. 4a, 4b and 4c. The resistive spacer 640 comprises an attachment for a calibration port 617, and/or has a calibration port 617 formed integrally therewith. If the natural orifice is a patient's mouth, the resistive spacer 640 may be configured to bridge the patient's head so as to bridge the mouth. The resistive spacer shown in FIG. 6a is a frame comprising four legs. In use, two of the legs may be positioned on the operating table on one side of the patient's head, with the other two legs positioned on the operating table on the other side of the patient's head. Although the resistive spacer shown in FIG. 6a comprises four legs, it is to be understood that the resistive spacer in accordance with the principles described herein may comprise more or fewer legs. The legs may be attached to the operating table, e.g. using clamps.

The dimensions of the resistive spacer 640 shown in FIG. 6a are dictated by the dimensions of the part of the patient's body in which the natural orifice exists. For example, where the natural orifice is the mouth, the dimensions of the resistive spacer 640 will be dictated by the expected size of the patient's head. For example, the upper surface of the resistive spacer 640 may be square and have dimensions between 250 mm by 250 mm and 500 mm by 500 mm, and the height of the legs may be between 250 mm and 500 mm. In one example, the upper surface of the resistive spacer 640 may have dimensions of 300 mm by 300 mm, and the height of the legs may be 250 mm. In other examples, the upper surface of the resistive spacer 640 may be rectangular as the resistive spacer may span the width of the head in a first direction such that the legs can rest on the operating table, but need not necessarily span the length of the head in a second direction. For example, a rectangular resistive spacer could have a dimension in the first direction in the range 250 mm to 500 mm and a dimension in second direction in the range of 150 mm to 500 mm, and the height of the legs may be between 250 mm to 500 mm. In one example, the upper surface of the resistive spacer 640 may have dimensions of 300 mm by 200 mm, and the height of the legs may be 250 mm. The upper surface of the resistive spacer 640 may be any other shape, such as circular, oval, hexagonal, or any other suitable shape. The height of the legs could be adjustable so as to vary the distance between the natural orifice and the calibration port 617.

The resistive spacer 640 is capable of resisting external forces applied to the calibration port by the surgical instrument such that, during the calibration process, the calibration port is maintained in the fixed position. That is, as described herein, the rigid linear shaft of the surgical instrument interacts with and exerts forces onto the passageway of the port whilst it is being gyrated during the calibration process. Said forces can be in the region of 2 to 3 Newtons. The resistive spacer 640 is capable of resisting external forces of at least 2 Newtons applied to the calibration port by the surgical instrument. In order to achieve this, the resistive spacer may be constructed from a material capable of resisting said forces, such as surgical steel.

In an example, the resistive spacer 640 may comprise an attachment for a calibration port 617 that is movably mounted with respect to the resistive spacer 640. The calibration port attachment may be mounted on one or more moveable rails 680 such that, when the resistive spacer is bridging the natural orifice, the calibration port attachment is moveable in a plane above the natural orifice. For example, rail 680 may be movable with respect to the frame in direction 682. Direction 682 is a Y-direction. Rail 680 may slide in a pair of parallel grooves on opposing sides of frame 640. The calibration port attachment may be moveable with respect to rail 680 in direction 681. Direction 681 is an X-direction, and is in the same plane and perpendicular to Y-direction 682. The calibration port attachment may slide in a groove in rail 680. Alternatively, the rail 680 may comprise a plurality of apertures in positions along direction 681 in which a calibration port can be received. The calibration port attachment is securable in position relative to the resistive spacer such that, in use, the calibration port 617 is held in a fixed position. For example, the rail and calibration port attachments may comprise respective screws that can be tightened so as to impinge on the grooves in which they are mounted, so as to prevent sliding. The resistive spacer 640 may comprise multiple pairs of parallel grooves at different heights in which the rail 80 could slide (e.g. see groove 685a and groove 685b in the expanded region of FIG. 6a). In this way, the distance between the natural orifice and the attachment for a calibration port 617 can be varied depending on which pairs of parallel grooves are selected. Optionally, the orientation of the calibration port may also be variable. By way of these features, the surgeon or member of operating room staff can position the calibration port in a desirable position above the natural orifice such that the end effector of the surgical instrument could be inserted into the natural orifice via the calibration port when the calibration port is held in the resistive spacer 640.

In another example, the resistive spacer 640 may have a calibration port 617 formed integrally therewith that is moveable with respect to the resistive spacer 640. For example, the calibration port may be formed integrally with a moveable rail (e.g. having the same features as rail 680 described herein) such that, when the resistive spacer is bridging the natural orifice, the calibration port is moveable in a plane above the natural orifice. The calibration port may be securable in position relative to the resistive spacer such that, in use, the calibration port is retained in the fixed position.

The position of the calibration port with respect to the resistive spacer may be variable prior to the calibration process for a surgical instrument, and securable for the duration of calibration process for that surgical instrument. The position of the calibration port may be changed after calibrating a first surgical instrument so as enable a second surgical instrument to be calibrated using the same calibration port.

The resistive spacer 640 may comprise multiple calibration port attachments and/or integrally formed calibration ports such that multiple surgical robotic arms each comprising a surgical instrument to be used in the minimally invasive procedure can be calibrated using the same surgical robot calibration device (e.g. theoretically at the same time). This can be advantageous as it enables multiple surgical robotic arms to be arranged with respect to each other about the operating table and calibrated without “losing” the desired calibration position by re-positioning a single calibration port between calibrating each surgical robotic arm.

The calibration process could be performed with the surgical instrument inserted into the surgical robot calibration device only so far that the end effector is in the empty space between the resistive spacer 640 that bridges the natural orifice and the natural orifice. In this way, the movement of the instrument during the calibration process does not cause the end effector of the surgical instrument to impinge on the interior of the natural orifice—and so damage to the natural orifice can be avoided.

After the calibration process has been completed the rail 680, including the attachment for the calibration port 617 or the integrally formed calibration port 617 could be removed from the resistive spacer 640. Alternatively, the entire resistive spacer 640 may be removed. The resistive spacer 640 may be sterilisable and re-usable.

FIG. 6b shows a seventh example surgical robot calibration device. In this example, the resistive spacer 640 is configured to bridge the natural orifice as described with reference to FIG. 6a. Relative to the sixth example shown in FIG. 6a, the resistive spacer 640 shown in FIG. 6b comprises a surface 641 that is pierceable so as to receive a calibration port 617. That is, the surface 641 may initially comprise no apertures configured to receive a calibration port, but may be pierceable by a process such as cutting or drilling so as to create an aperture for receiving a calibration port 617. In this way, a surgeon or member of operating room staff can determine the precise position in which the calibration port 617 is to be held, rather than selecting from one or more predetermined positions. In other words, the resistive spacer 640 is configurable to hold a calibration port 617 in a fixed position spaced from the natural orifice such that the end effector of the surgical instrument could be inserted into the natural orifice via the calibration port when the calibration port is held in the resistive spacer 640.

Alternatively, or additionally, the surface 641 may comprise one or more apertures (not shown) each configured to receive a calibration port (e.g. as in the surfaces 441 described with reference to FIGS. 4b and 4c). Alternatively, or additionally, the surface may have one or more calibration ports formed integrally therewith (e.g. as described with reference to FIGS. 4e and 4f).

The surface 641 may be constructed from a surgical mesh or a silicone film of sufficient thickness so as to resist external forces applied to the calibration port by the surgical instrument during the calibration process.

After the calibration process has been completed the surface 641 could be removed from the frame of the resistive spacer 640. Alternatively, the entire resistive spacer 640 may be removed. The frame may be sterilisable and re-usable. The surface 641 may be sterilisable and re-usable, or may be disposable after a single use.

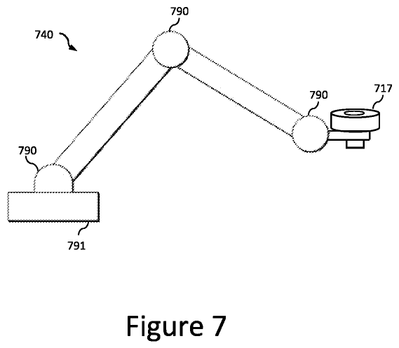

FIG. 7 shows a eighth example surgical robot calibration device. The surgical robot calibration device comprises a resistive spacer 740. The resistive spacer 740 extends from a base 791 at its proximal end. The base may be attached to the operating table, e.g. by one or more clamps. The resistive spacer 740 comprises a plurality of joints 790 by which the configuration of that resistive spacer 740 can be altered. The resistive spacer comprises an attachment for a calibration port 717 at its distal end, and/or has a calibration port 717 formed integrally therewith at its distal end. Using the resistive spacer depicted in FIG. 7 the surgeon or member of operating room staff can alter the configuration of the resistive spacer so as to position the calibration port in a desirable position above the natural orifice such that the end effector of the surgical instrument could be inserted into the natural orifice via the calibration port when the calibration port is held in the resistive spacer 740. Optionally, the orientation of the calibration port may also be variable. The joints 790 of the resistive spacer are securable in position such that, in use, the calibration port 717 is held in a fixed position. For example, each joint may comprise a screw that can be tightened so as to impinge on two parts of the joint so as to prevent relative movement between those parts.

The resistive spacer 740 is capable of resisting external forces applied to the calibration port by the surgical instrument such that, during the calibration process, the calibration port is maintained in the fixed position. That is, as described herein, the rigid linear shaft of the surgical instrument interacts with and exerts forces onto the passageway of the port whist it is being gyrated during the calibration process. Said forces can be in the region of 2 to 3 Newtons. The resistive spacer 740 is capable of resisting external forces of at least 2 Newtons applied to the calibration port by the surgical instrument. In order to achieve this, the resistive spacer may be constructed from a material capable of resisting said forces, such as surgical steel.

The calibration process could be performed with the surgical instrument inserted into the surgical robot calibration device only so far that the end effector is in the empty space between the resistive spacer 740 and the natural orifice. In this way, the movement of the instrument during the calibration process does not cause the end effector of the surgical instrument to impinge on the interior of the natural orifice—and so damage to the natural orifice can be avoided.

In another example, not shown within the Figures, the resistive spacer of the surgical robot calibration device may be a surgical robotic arm (such as robotic arm 201 shown in FIG. 2) comprising an attachment for a calibration port and/or having a calibration port formed integrally therewith. In a further example, not shown within the Figures, the resistive spacer of the surgical robot calibration device may be a surgical robotic arm (such as robotic arm 201 shown in FIG. 2) to which a resistive structure configurable to hold a calibration port, or a resistive structure having a calibration port formed integrally therewith, has been attached.

The surgical robot and surgical robot calibration device described herein could be used for purposes other than surgery. For example, the robot could be used to control a viewing instrument for inspecting a manufactured article, such as the inside of a car engine, and the robot calibration device could be used to calibrate that robot prior to performing the inspection procedure.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A surgical robot calibration device configured to be used when calibrating a surgical robotic system to perform a minimally invasive procedure through a natural orifice, the surgical robotic system comprising a surgical robotic arm and a surgical instrument having a rigid linear shaft, the surgical robot calibration device comprising a resistive spacer configurable to hold a calibration port in a fixed position spaced from the natural orifice, such that when the calibration port is held in the resistive spacer, the surgical instrument is insertable into the natural orifice via the calibration port to enable a fulcrum about which the surgical instrument pivots whilst the surgical instrument is inserted into the calibration port to be determined.

2. A surgical robot calibration device as claimed in claim 1, wherein the natural orifice is a mouth.

3. A surgical robot calibration device as claimed in claim 1, wherein the resistive spacer is configured to be received at one end in the natural orifice, and comprises an aperture at an opposing end for receiving the calibration port.

4. A surgical robot calibration device as claimed in claim 1, wherein the resistive spacer is configured to be received at one end in the natural orifice and comprises a surface spaced from that end having one or more apertures each configured to receive a calibration port.

5. A surgical robot calibration device as claimed in claim 1, wherein the resistive spacer is configured to be received at one end in the natural orifice and comprises a surface spaced from that end that is pierceable so as to receive a calibration port.

6. A surgical robot calibration device as claimed in claim 4, wherein the resistive spacer is curved so as to define a substantially domed three-dimensional shape.

7. (canceled)

8. (canceled)

9. (canceled)

10. (canceled)

11. (canceled)

12. (canceled)

13. (canceled)

14. A surgical robot calibration device as claimed in claim 1, wherein the resistive spacer extends from a base at its proximal end, comprises a plurality of joints by which the configuration of that resistive spacer can be altered and comprises an attachment for the calibration port at its distal end.

15. A surgical robot calibration device configured to be used when calibrating a surgical robotic system to perform a minimally invasive procedure through a natural orifice, the surgical robotic system comprising a surgical robotic arm and a surgical instrument having a rigid linear shaft, the surgical robot calibration device comprising a resistive spacer having formed integrally therewith a calibration port, the resistive spacer being configured to retain the calibration port at a fixed position spaced from the natural orifice, such that the surgical instrument is insertable into the natural orifice via the calibration port to enable a fulcrum about which the surgical instrument pivots whilst the surgical instrument is inserted into the calibration port to be determined.

16. (canceled)

17. A surgical robot calibration device as claimed in claim 15, wherein the resistive spacer is configured to be received at one end in the natural orifice, and the calibration port is formed integral therewith at an opposing end.

18. A surgical robot calibration device as claimed in claim 15, wherein the resistive spacer is configured to be received at one end in the natural orifice and comprises a surface spaced from that end having one or more calibration ports formed integral therewith.

19. A surgical robot calibration device as claimed in claim 18, wherein the resistive spacer is curved so as to define a substantially domed three-dimensional shape.

20. (canceled)

21. (canceled)

22. (canceled)

23. (canceled)

24. (canceled)

25. (canceled)

26. A surgical robot calibration device as claimed in claim 15, wherein the resistive spacer extends from a base at its proximal end, comprises a plurality of joints by which the configuration of that resistive spacer can be altered and has the calibration port formed integrally therewith at its distal end.

27. A surgical robot calibration device as claimed in claim 15, wherein the natural orifice is a mouth and the resistive spacer comprises an attachment for a mouth retractor.

28. A surgical robot calibration device as claimed in claim 27, wherein the mouth retractor is detachable from the attachment.

29. A surgical robot calibration device as claimed in claim 28, wherein the resistive spacer is elastically deformable, and the attachment comprises a lip, such that:
with the resistive spacer in a first configuration, the lip attaches the mouth retractor to the resistive spacer; and
with the resistive spacer in a second, deformed, configuration, the mouth retractor can pass over the lip in order to detach the mouth retractor from the resistive spacer.

30. A surgical robot calibration device as claimed in claim 1, wherein the resistive spacer is capable of resisting external forces applied to the calibration port by the surgical instrument such that, in use, the calibration port is maintained in the fixed position.

31. (canceled)

32. A method of calibrating a surgical robotic system to perform an invasive procedure via a natural orifice using the surgical robot calibration device as claimed in any preceding claim, the surgical robotic system comprising a surgical robotic arm having a series of joints by which the configuration of the robotic arm can be altered, the series of joints extending from a base at a proximal end of the surgical robotic arm to a surgical instrument having a rigid linear shaft attached at a distal end of the surgical robotic arm, and the method comprising determining a fulcrum about which the surgical instrument pivots when the configuration of the robotic arm is altered whilst the surgical instrument is inserted into the calibration port.

33. A surgical robot calibration device as claimed claim 1, wherein the natural orifice is a mouth and the resistive spacer comprises an attachment for a mouth retractor.

34. A surgical robot calibration device as claimed in claim 33, wherein the mouth retractor is detachable from the attachment.

35. A surgical robot calibration device as claimed in claim 34, wherein the resistive spacer is elastically deformable, and the attachment comprises a lip, such that:
with the resistive spacer in a first configuration, the lip attaches the mouth retractor to the resistive spacer; and
with the resistive spacer in a second, deformed, configuration, the mouth retractor can pass over the lip in order to detach the mouth retractor from the resistive spacer.
